(19) United States
(12) Patent Application Publication
(10) Pub. No.: US 2009/0264728 A1
(43) Pub. Date: Oct. 22, 2009

(54) BIOLOGY GUIDED ADAPTIVE THERAPY PLANNING

(75) Inventors: Alexander Fischer, Aachen (DE); Lothar Spies, Hamburg (DE)

Correspondence Address: PHILIPS INTELLECTUAL PROPERTY & STANDARDS, P. O. Box 3001, BRIARCLIFF MANOR, NY, 10510, US

(73) Assignee: KONINKLIJKE PHILIPS ELECTRONICS N. V., Eindhoven (NL)

(21) Appl. No.: 12/375,430

(22) PCT Filed: Jul. 23, 2007
(86) PCT No.: PCT/US07/74077

§ 371 Date: Jan. 28, 2009

Related U.S. Application Data
(60) Provisional application No. 60/820,964, filed on Aug. 1, 2006.

(51) Int. Cl. A61B 6/00; A61N 5/00

(52) U.S. Cl. 600/407

(57) ABSTRACT

A therapy system (100) includes an imager (102), a therapy planner (104), and a therapy device (106). The therapy planner (104) includes a therapy prescription apparatus (118) which calculates a desired therapy (D) to be applied to a human patient or other subject. The therapy prescription system (118) uses a pathology model (122) and a patient-specific biological parameter history (124) to optimize the applied therapy.

The present application relates to therapy planning in medicine. While it finds particular application to external radiotherapy and molecular therapeutics, it also relates to other situations in which a therapy is applied to a patient or other subject.

Computed tomography (CT) images are widely used in connection with radiotherapy therapy planning (RTP) in oncology. To develop a therapy plan, the tumor and risk organs are located and delineated in the CT images, and suitable dose levels are prescribed. The prescribed therapy plan is ordinarily designed to maximize the radiation dose applied to the target tissue while minimizing the damage to surrounding tissue and risk organs.

In fractionated radiotherapy, the prescribed dose is applied in fractions over a desired time period, for example over the course of a few weeks. The fractionation allows the healthy tissue to recover at least partially from the unwanted radiation effects. Consequently, a higher total dose may be applied to the target tissue compared to what could ordinarily be applied in a single application.

Conventionally, a fractionated therapy plan is applied to the patient by registering the radiation beam with respect to artificial or natural fiducial markers (such as tattoos or other applied markers, bones and other anatomical structures, or the like) having a known relation to the target region. However, factors such anatomical changes and changes to the markers between treatment fractions and patient motion during a given treatment fraction can cause misregistration and other positioning errors. As a result, the realized exposure may differ from the therapy plan.

Image guided or adaptive radio therapy (ART) techniques reduce such discrepancies by applying image-based corrections to the fractionated treatments. As a consequence, the applied dose can be tailored to more closely match that of the initially calculated plan. See Erbel et al., Method for creating or updating a radiation treatment plan, European patent application EP1238684 (2005); Ruchala et al., Method for modification of radiotherapy treatment delivery, United States patent publication 20050201516 (2005); Amies et al., Active therapy redefinition, United States patent publication 20040254448 (2004); Rehbinder, et al., Adaptive radiation therapy for compensation of errors in patient setup and treatment delivery, Med Phys. vol. 31, no. 12, pp. 3363-3371 (2004); Lam, et al., An application of bayesian statistical methods to adaptive radiotherapy, Phys Med Biol. vol. 50, no. 16, pp. 3849-3858 (August 2005); Schaly, et al., Image-guided adaptive radiation therapy (igart): Radiobiological and dose escalation considerations for localized carcinoma of the prostate, Med Phys. vol. 32, no. 7, pp. 2193-2203 (July 2005); Yan, et al., Computed tomography guided management of interfractional patient variation, Seminars in Radiation Oncology, vol. 15, no. 3, pp. 168-179 (July 2005).

In contrast to ART, biology guided radiotherapy (BGRT) takes advantage of functional imaging techniques which provide information on metabolic parameters. By using a priori knowledge of suitable functional parameters, a therapy plan which optimizes the expected therapeutic impact on the target tissue is calculated. See Xing et al., Inverse planning for functional image-guided intensity-modulated radiation therapy, Phys Med Biol. vol. 47, pp. 3567-3578 (2002). The calculated therapy plan is then applied on a fractioned basis and otherwise.

While ART and BGRT techniques have been successfully used in the treatment of disease, there remains room for improvement. More particularly, it is desirable to tailor the therapy plan to account for biological variations in a particular pathology or patient.

Aspects of the present invention address these matters and others.

In accordance with one aspect, a therapy prescription apparatus uses a mathematical pathology model and a subject-specific biological parameter history to establish a desired therapy to be applied to the subject. The pathology model models a response of a pathology to a therapy and the biological parameter history includes a biological parameter value obtained from a functional imaging examination of the subject.

According to another aspect of the present invention, a therapy prescription method includes using a mathematical pathology model and a subject-specific biological parameter history to establish a desired therapy to be applied to the subject. The pathology model models a response of a pathology to a therapy and the biological parameter history includes spatially varying biological parameter values obtained from a functional imaging examination of the subject.

According to another aspect, a therapy prescription apparatus calculates a therapy (D) to be applied to a pathology based on a desired biological parameter value, measured values of the biological parameter (b_{i,measured}), and a mathematical pathology model (122) which models the response of a pathology to a therapy. The biological parameter is measured following the application of a therapy to the pathology and the measured values include spatially varying biological parameter values.

According to another aspect of the invention, a computer readable storage medium contains instructions which, when carried out by a computer, cause the computer to carry out a method which includes using a desired biological parameter value, a subject-specific measured biological parameter history, and a mathematical pathology model to establish a desired therapy to be applied to a pathology of the subject.

According to another aspect of the invention, an apparatus includes a therapy planning system and a therapy device. The therapy system establishes a characteristic of successive therapies applied to a subject as a function of a desired biological parameter value of the subject, a subject specific biological parameter history indicative of a pathology of the subject, and a pathology model which models a response of the pathology to a therapy. The therapy device is operatively electrically connected to the therapy planning system so as to receive the established characteristic and applies a therapy according to the established characteristic.

According to another aspect, a method includes obtaining data representative of a measured response of a patient population to an applied therapy, storing the data in a computer readable storage medium, and making the data available over a therapy planning system over a computer network. The data includes a measured biological parameter value, the applied therapy, and a second measured biological parameter value representative of a response to the applied therapy. The first and second measured biological parameter values are obtained from functional imaging examinations of members of the subject population.

Still further aspects of the present invention will be appreciated by those of ordinary skill in the art upon reading and understanding the following detailed description.

The invention may take form in various components and arrangements of components, and in various steps and arrangements of steps. The drawings are only for purposes of illustrating the preferred embodiments and are not to be construed as limiting the invention.

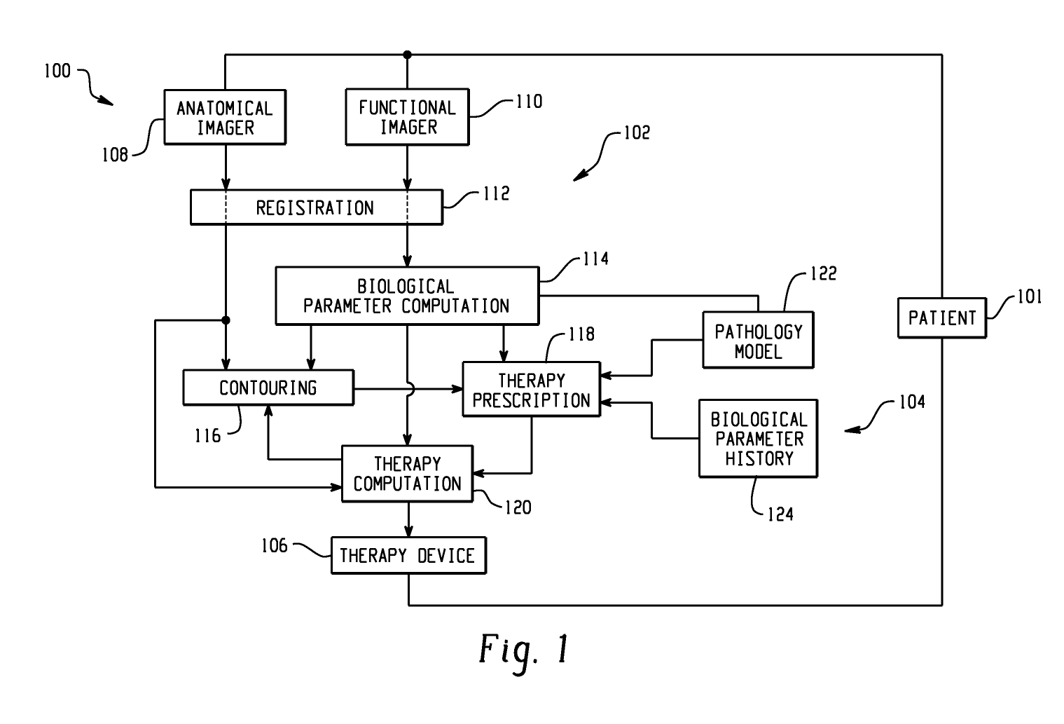

FIG. 1 depicts a therapy planning system.

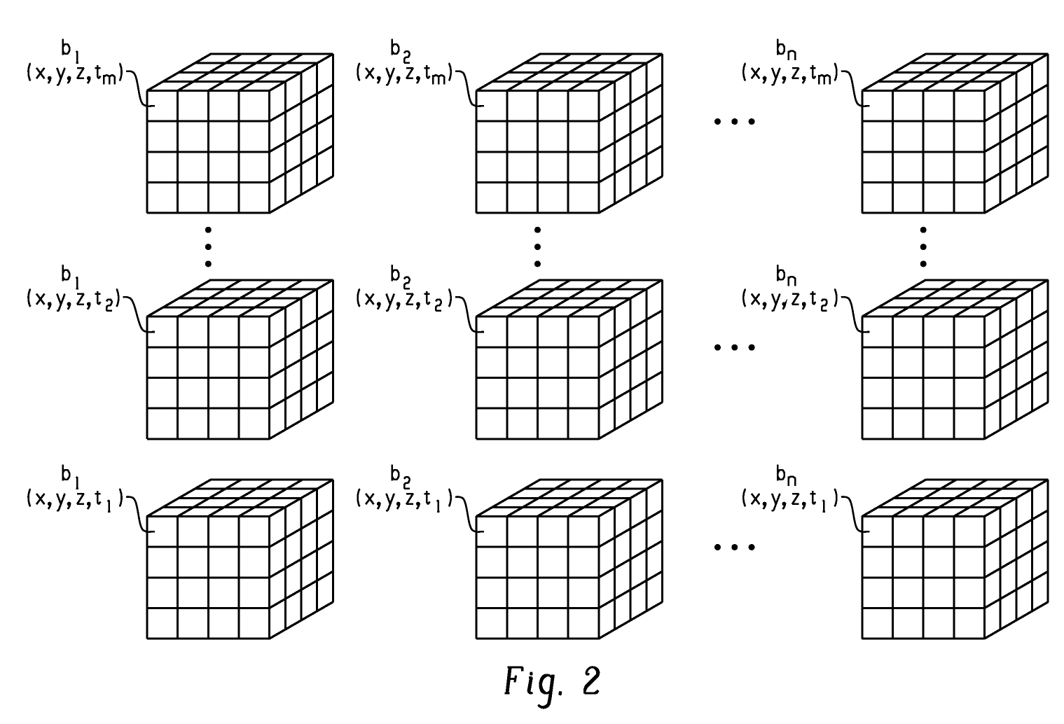

FIG. 2 depicts a biological parameter history.

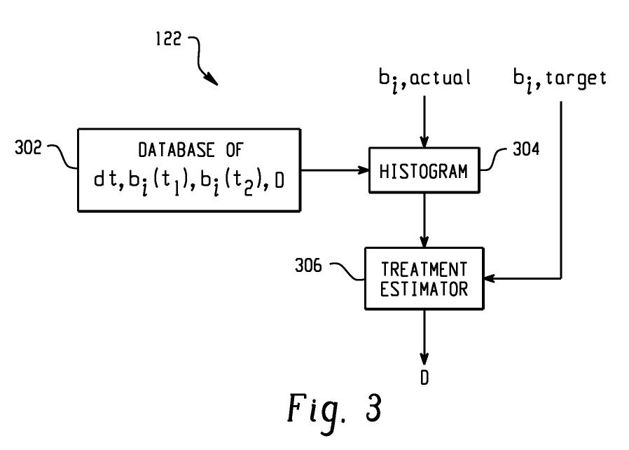

FIG. 3 depicts a pathology model.

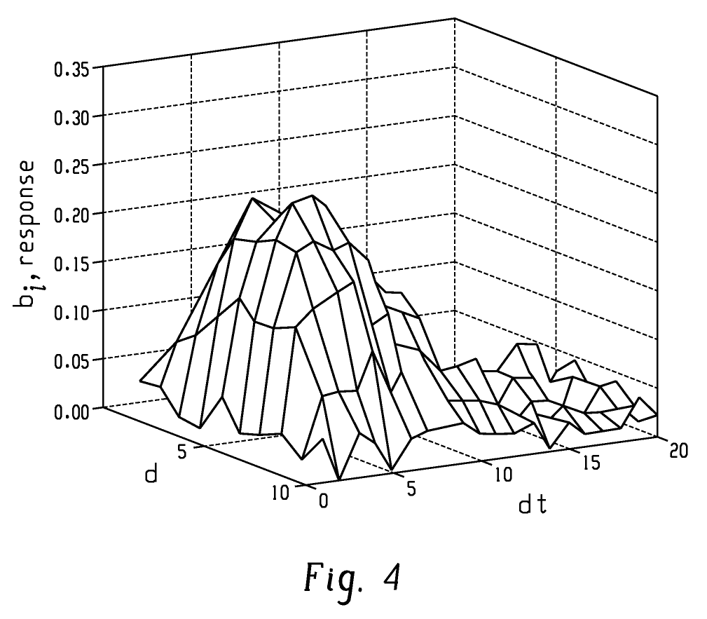

FIG. 4 depicts predicted responses to a therapy.

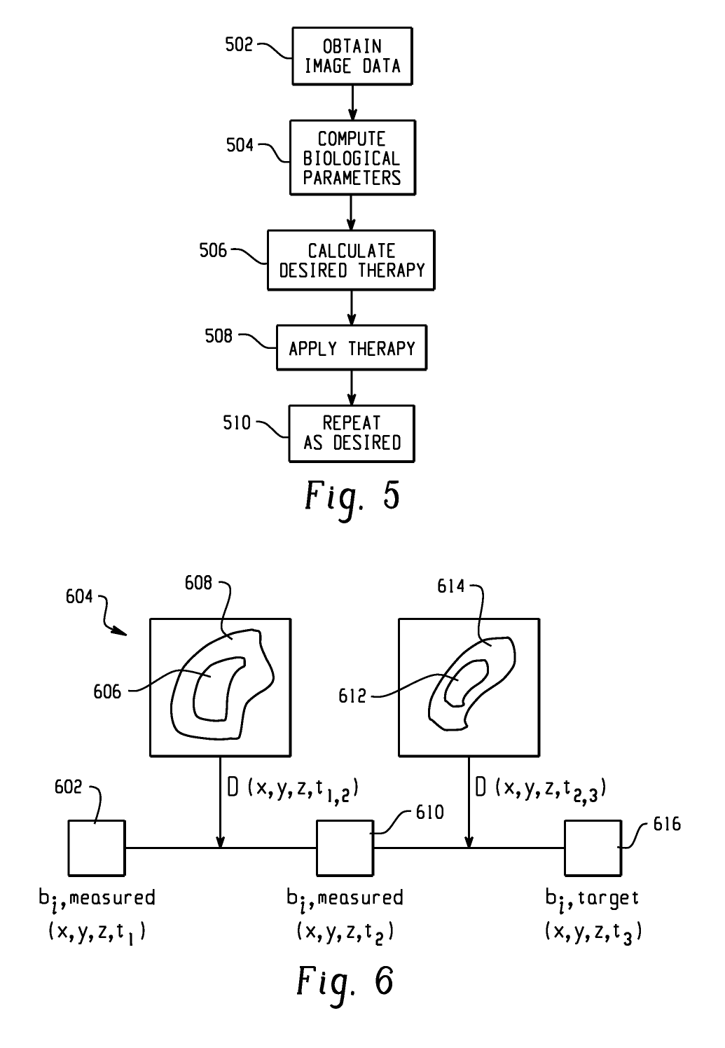

FIG. 5 depicts a therapy method.

FIG. 6 depicts a therapy method.

With reference to FIG. 1, a biology guided adaptive radiotherapy (BGART) system 100 includes an imager 102, an adaptive therapy planning system 104, and a therapy device 106.

The imager 102 includes an anatomical imager 108 and a functional imager 110. The anatomical imager 108 is of an anatomical imaging modality such as a computed tomography (CT), magnetic resonance (MR), x-ray, fluoroscopic or other scanner which provides anatomical information representative of a patient or subject 101. The functional imager 110 is of a functional imaging modality such as a positron emission tomography (PET), single photon emission computed tomography (SPECT), functional MR (fMR), or other scanner which provides functional information. The imager 102 also includes a registration unit 112 which registers or correlates the volumetric data generated by the anatomical 108 and functional 110 imagers, for example to account for gross and periodic patient motion.

In one implementation, the imager 102 is a hybrid scanner such as a hybrid PET/CT, SPECT/CT, PET/MR, or SPECT/MR system. In such hybrid systems, two or more modalities typically share a common gantry structure or are otherwise located in close proximity to each other, for example with their respective examination regions being at least partially overlapping or disposed along a common longitudinal axis. To reduce the need for repositioning the patient between scans, hybrid systems typically share a patient support which can be used to variously position the patient in the respective examination regions as required.

With continuing reference to FIG. 1, the adaptive therapy planning system 104, which is operatively electrically connected to the imager 102, includes biological parameter computation 114, contouring 116, therapy prescription 118, and dose calculation 120 subsystems.

The biological parameter computation subsystem 114 uses information from the functional imager 110 to generate one or more biological parameter maps representative of a biological parameter or parameters of a region of interest of the subject. In the case of oncology, for example, typical biological parameters may include the radiosensitivity (e.g., as obtained from a PET scan using a tracer such as FMISO) or proliferation (e.g., as obtained from a PET scan using a tracer such as FLT) of a tumor. Other biological parameters are also contemplated, depending on the characteristics of a particular functional imager 110 and tracer, as well as other application specific requirements.

For the purpose of the following discussion, the various biological parameters will be termed bi, where i=1, 2, 3 . . . N. While improved spatial accuracy is generally obtained by modeling the biological and other parameters at the voxel level, the modeling may be performed at a desired level of granularity, depending on the required accuracy, the characteristics of the imager 102, and other application specific factors.

The contouring subsystem 116 uses information from the anatomical imager 108 and/or the biological parameter computation subsystem 120 to delineate one or more regions of interest in the image data. Thus, for example, the contouring system may delineate one or more pathologic regions such as a tumor or other lesion which requires treatment. The contouring subsystem 116 may also delineate one or more regions of healthy tissue for which treatment should be avoided.

The biology adaptive therapy prescription subsystem 118 uses information from the biological parameter computation 114 and contouring 116 subsystems to calculate a desired therapy D. In the exemplary case of radiation oncology, the desired therapy D may include a target dose map which indicates a desired radiation dose to be applied to one or more regions of a tumor, as well as a desired time between therapy fractions. The desired therapy D may also provide maximum dose information for or otherwise delineate healthy areas which should be spared treatment.

As will be described in more detail below, the therapy prescription system 118 also applies a pathology model 122 and biological parameter history information 124 to adapt or otherwise tailor the therapy based on the observed characteristics of a particular patient or pathology, for example based on the response of the pathology or adjacent healthy tissue to previously applied treatments.

The therapy computation subsystem 120 uses the desired therapy D from the prescription subsystem 118 in combination with anatomical, biological, contour and/or other data to calculate a therapy plan which approximates the target therapy. In the case of a radiation oncology application where the therapy is to be carried out using an external radiotherapy device, the therapy computation subsystem 120 uses known intensity modulated radiation therapy (IMRT) or other techniques to calculate one or more desired beam paths, exposure times, and similar information so that the spatial distribution of the applied radiation dose approximates the target dose map.

The therapy device 106, which communicates with the therapy planning system 104 over an electrical or other network communication interface, applies the desired therapy D to the patient or subject. While the above discussion has focused on radiation oncology and the use of an external radiotherapy device, it should be understood other external and non-external therapy devices 106 are contemplated and may be selected depending on factors such as relevant pathology and the desired treatment modality. Non-limiting examples of such therapy devices include brachytherapy, high intensity focused ultrasound (HIFU), and thermal and/or radiofrequency ablation, cryotherapy, and surgical devices, as well as molecular or chemical (e.g., chemotherapy) therapeutics.

The biology adaptive therapy prescription subsystem 118 will now be described in greater detail. As noted above, the prescription subsystem 118 applies a pathology model 122 and biological parameter history information 124 to tailor treatment according to the characteristics of a particular patient or pathology. While it is generally desirable that the pathology model 122 model the transfer function of the biological system as precisely as possible, those of ordinary skill in the art will appreciate that the model 122 is likely be imperfect. These imperfections can arise from a number of factors, such as the number and selection of the (measurable) parameters, patient and pathology specific variations, and like factors.

Viewed from one perspective, then, the therapy prescription subsystem 118 can be viewed as implementing part of an iterative or closed loop system which receives the actual b_{i,actual} and desired b_{i,target} values of the relevant biological parameter(s) bᵢ as inputs. The therapy prescription system 118 uses the pathology model 122 and the biological parameter history information 124 to adjust the therapy so that the actual biological parameter value(s) b_{i,actual} approximate the desired parameter value(s) b_{i,target} value(s). Again, the actual b_{i,actual} and desired b_{i,target} parameter values may be modeled at the voxel level or other desired level of granularity.

The turning now to FIG. 2, the biological parameter history 124 can be visualized as a multidimensional matrix containing the values of one more biological parameters bᵢ as measured at one or more times tₘ, for example at various times during the course of a fractionated therapy applied to a given patient. Those of ordinary skill in the art will recognize that while FIG. 2 presents the biological parameter history 124 in a manner convenient for illustration, the history 124 may be organized in any suitable data structure, for example in a computer readable memory.

Turning now to FIG. 3, the pathology model 122 receives one or more measured b_{i,actual} and desired b_{i,target} biological parameter values as inputs and generates an output which includes the desired therapy D. As shown in greater detail in FIG. 3, an exemplary empirical pathology model 122 includes a database 302, a histogram 304, and a treatment estimator 306.

The database 302, which can be viewed as providing information on the expected response to and/or the effectiveness of an applied therapy for members of a given subject population, includes measured biological parameters b_{i,actual} and prescribed therapies D obtained from a plurality of cases. As illustrated, the database 302 includes a series of entries of the form:

dt, bᵢ(t₁), bᵢ(t₂), D_{applied} Equation 1

where bᵢ(t₁) is the measured value of biological parameter bᵢ at a time t₁, bᵢ(t₂) is the measured value of the biological parameter bᵢ at a time t₂, and D_{applied} is the applied therapy. In the case of a fractionated therapy, D_{applied} can represent a list of applied dose fractions and times. The database entries may also contain additional or different information such as age and other patient demographic data, pathology location, imaging agent, and other information which is expected to influence the response to a particular therapy.

Information can be extracted from the database 302 to provide more generalized information on the expected responses to the applied therapy D. As one example, the information can be used to generate a conditional two-dimensional histogram of the form b_{i,response}(dt,D)|b_{i,initial}, where b_{i,response} represents the predicted value of biological parameter bᵢ at a time dt following application of therapy D, assuming an initial biological parameter value b_{i,initial}.

A illustrative example of an arbitrary two dimensional histogram is presented in FIG. 4. For an initial biological parameter b_{i,initial}, the histogram can be used to determine those combinations, if any, of doses d and time periods dt which can be expected to result in a target state b_{i,target}. As illustrated in FIG. 4, the possible combinations are disposed in a plane located at the desired biological parameter value b_{i,target}. Similarly, histogram peaks (or valleys, depending on the presentation of the data) can be used to identify those therapies D which are expected to have the maximum effect. While a two dimensional histogram is illustrated, histograms having three (3) or more dimensions may also be generated.

The biological parameter history 124 may also be used to further refine the selected therapy D. Thus, for example, the measured response of the particular patient to a previously applied therapy may be compared to the response predicted by the pathology model 122 and the selected therapy D adjusted accordingly. Where, for example, the particular patient has responded less favorably than predicted by the model 122, the applied dose may be adjusted upwardly.

The treatment estimator 306 receives the information from the histogram 304 and the desired target state b_{i,target} to select a therapy D which is estimated to provide the desired target state. Note that the target state b_{i,target} may be established based on the literature, the pathology model 122, operator experience, or other factors. Where the treatment estimator 306 identifies more than one possible therapy D, the treatment estimator 306 may suggest a suitable therapy based on a desired rule (e.g., minimum applied dose d, minimum expected time dt until the target state is reached) or request that the user select from among the possible therapies.

While the above has described one implementation of the system 100, variations are contemplated. For example, the imager 102 may be implemented as other than a hybrid imaging system. Thus, the anatomical 108 and functional 110 imagers may also be implemented as separate systems or as a single imager which can be used to obtain both anatomical and functional information, for example in the case of an fMR scanner. The anatomical imager 108 may also be omitted.

The therapy planning system 104 is advantageously implemented on a computer workstation such as a general purpose or other computer having a graphical user interface (GUI) for interacting with the user. The therapy planning system 104 may also be incorporated in a workstation associated with the imager 102, using multiple computers, or otherwise. The registration system may likewise be implemented separately from the imager 102, as part of the therapy planning system 104, or otherwise. It will be appreciated that the various computers contain or otherwise access computer readable storage media containing instructions which, when carried out the by the computer processor(s), cause the computers to carry out the described techniques.

While the above discussion of an empirically-based pathology model 122 was described in relation to a histogram 304, other suitable mathematical models may also be employed. Moreover, the pathology model 122 may also be radiobiologically or analytically based. In such a case the desired treatment D may be calculated using a suitable mathematical model. The pathology model 122 may also be rule based, for example in connection with an expert system based implementation.

The database 302 may also contain information on various alternative therapies, for example responses to more than one molecular agent. The database 302 may also contain information on various therapeutic modalities, for example information on the responses to radiation, molecular, thermal or other therapeutics, whether applied separately or as adjunct or otherwise supplemental therapies. Thus, the pathology model 122 may also model the response of the pathology to more than one treatment type and be used to suggest not only optimization of the current treatment, but also alternative or supplemental therapies. In this regard, a desired molecular agent or other therapeutic modality, dose level, or therapy interval may also be accepted as an input to the therapy determination. Information from the database 302 and/or the biological parameter history 124 may be used to display trends in the treatment plan.

The database 302 need not be stored on the therapy planning system 104. Indeed, the database itself 302 need not be accessible to the therapy prescription subsystem 118. In the latter case, the database 302 may be used to develop a suitable pathology model 122 which is in turn accessible to the planning system 104. In either case, the database 302, or information derived from the database may be stored in a computer readable memory accessible to the therapy planning system 104 or accessed over a network such as a hospital HIS/RIS system, a DICOM interface, the internet, or the like. The pathology model 122 may also be updated from time to time to reflect changes in the database 302. The database 302 may likewise be updated from time to time to reflect additional or different data.

Similarly, the biological parameter history 124 need not be stored on the therapy planning workstation. Rather, the desired information may be stored at a remote location and accessed as needed, for example over HIS/RIS system, DICOM interface, the internet, or other suitable communications network

Applications other than radiation oncology are also contemplated. For example, the described techniques are applicable to molecular therapeutics and chemotherapy. Still other applications will be appreciated by those skilled in the art.

Operation of the system 100 will now be described in relation to FIG. 5.

Functional information is acquired at step 502, for example using the functional imager 110. Desired anatomical information is likewise obtained, and the required registration, contouring, and similar steps are performed. The resultant image data is stored in the biological parameter history 124. Note that an initial image set is advantageously acquired prior to the initial therapy.

Information from the functional imager 110 is used to calculate the desired functional parameters bᵢ at step 504.

At step 506, the desired state(s) b_{i,target}, actual state(s) b_{i,actual}, and pathology model 122 are used to calculate the desired therapy D. Note that, in addition to a variation on a previously applied therapy, the desired therapy D may also suggest a change in the treatment plan, for example by suggesting a change from a molecular to a radiation therapy or to application of an adjunct or otherwise supplemental therapy. The user may be prompted to enter or otherwise confirm the target information b_{i,target}. Note that the target state need not be the final desired target state (e.g., a biological parameter value for substantially inactive tumor in the case of an oncology application), but may instead be an intermediate target state. In the case of a fractionated therapy, for example, the intermediate target state may be dependent on the current treatment fraction, thereby applying a therapy-fraction dependent moving target. Note that the target is advantageously selected at a condition which can be expected to be reached using an otherwise reasonable or appropriate set of dose, therapy interval, or other therapeutic parameters. The actual state information b_{i,actual} is advantageously obtained from the biological parameter history 124. The user may also be prompted to confirm or otherwise accept the proposed therapy D.

The therapy D is applied at step 508. In this regard, it should be noted that the therapy may include the application of one or more dose fractions.

The process is repeated as desired at step 510, for example until the pathology reaches the desired target state(s) b_{i,target}. As will be appreciated, such an iterative strategy helps to reduce the impact of imperfections in the pathology model 122. Moreover, information from subsequent measurements can be used to adapt the therapy to more closely reflect the actual response of the particular patient to the applied treatment.

A suitable therapy technique will be further described in relation to FIG. 6.

At 602, an initial biological parameter measurement b_{i,measured}(x,y,z,t₁) is obtained at time t₁. While illustrated at the voxel level, it will be appreciated that similar measurements are obtained for a plurality of voxels in the image space. Again, however, the measurements may also be obtained at other levels of granularity.

A first spatially varying therapy D(x,y,z,t_{1,2}) is calculated and applied at 604. In the illustrated example, a first dose is applied over a first spatial region 606, while a second dose is applied over a second spatial region 608. Again, however, the desired dose may be calculated and/or varied at the voxel or other desired level.

At 610, a second biological parameter measurement b_{i,measured}(x,y,z,t₂) is obtained at a desired time t₂ and compared against the goal state b_{i,target}(x,y,z).

If needed, a second spatially varying therapy D(x,y,z,t_{1,2}) is calculated and applied at 606. As illustrated, therapy prescription subsystem 118 varies the spatial extents and dose levels 612, 614 of the applied therapy based on the pathology model 122 and/or the biological parameter history 124.

At 616, a third biological parameter measurement b_{i,measured}(x,y,z,t₂) is obtained at a desired time t₂ and compared against the goal state b_{i,target}(x,y,z). The process may be continued as desired until the pathology reaches the goal state b_{i,target}(x,y,z).

The invention has been described with reference to the preferred embodiments. Modifications and alterations may occur to others upon reading and understanding the preceding detailed description. It is intended that the invention be construed as including all such modifications and alterations insofar as they come within the scope of the appended claims or the equivalents thereof.

1. A therapy prescription apparatus which uses a pathology model and a subject-specific biological parameter history to establish a desired therapy to be applied to the subject, wherein the pathology model models a response of a pathology to a therapy and the biological parameter history includes a biological parameter value obtained from a functional imaging examination of the subject.

2. The apparatus of claim 1 wherein the apparatus iteratively adjusts the desired therapy based on an observed response to an applied therapy.

3. The apparatus of claim 2 wherein the apparatus iteratively adjusts the desired therapy until a measured biological parameter value reaches a desired value.

4. The apparatus of claim 1 wherein the pathology model is an empirically derived model indicative of the responses of a subject population to a therapy of the type to be applied.

5. The apparatus of claim 1 wherein the pathology model models a change in the biological parameter value as a function of time.

6. The apparatus of claim 1 wherein the subject-specific biological parameter history includes a plurality of spatially varying biological parameter values.

7. The apparatus of claim 1 including a functional imager.

8. The apparatus of claim 1 including a therapy computation apparatus which computes a therapy to be applied by a therapy device.

9. The apparatus of claim 8 including a therapy device in operative electrical communication with the therapy computation apparatus.

10. (canceled)

11. The apparatus of claim 1 including a graphical user interface which presents information indicative of the desired therapy to a human user.

12. A therapy prescription method including using a pathology model and a subject-specific biological parameter history to establish a desired therapy to be applied to the subject, wherein the pathology model models a response of a pathology to a therapy and the biological parameter history includes spatially varying biological parameter values obtained from a functional imaging examination of the subject.

13. The method of claim 12 including
obtaining a biological parameter value from a functional imaging examination of the subject conducted after an application of the desired therapy; and
repeating the step of using a pathology model.

14. The method of claim 12 including comparing the obtained biological parameter value to a desired value.

15. The method of claim 12 wherein the desired therapy includes a dose and a treatment interval.

16. The method of claim 12 wherein the desired therapy includes a fractionated therapy.

17. The method of claim 12 wherein the desired therapy includes the application of thermal, radio frequency, or sonic energy.

18. The method of claim 12 wherein the desired therapy includes a molecular therapy or a chemotherapy.

19. The method of claim 12 wherein the pathology model includes an analytical model.

20. The method of claim 12 wherein the pathology model includes a multi-dimensional histogram.

21. The method of claim 12 including communicating information indicative of the desired therapy to a therapy device over an electrical communication interface.

22. A therapy prescription apparatus which calculates a therapy to be applied to a pathology based on:
a desired biological parameter value;
measured values of the biological parameter; and
a pathology model which models the response of a pathology to a therapy,
wherein the biological parameter is measured following the application of a therapy to the pathology and the measured values include spatially varying biological parameter values.

23. The apparatus of claim 22 wherein the therapy includes an external radiotherapy.

24. The apparatus of claim 22 wherein the calculated therapy includes a spatially varying dose.

25. The apparatus of claim 22 wherein the apparatus calculates the therapy based on a patient specific biological parameter history.

26. (canceled)

27. (canceled)

28. The apparatus of claim 22 including a computer readable memory containing data indicative of the responses of a patient population to a therapy of the type to be applied.

29. A computer readable storage medium containing instructions which, when carried out by a computer, cause the computer to carry out a method which includes using a desired biological parameter value, a subject-specific measured biological parameter history, and a pathology model to establish a desired therapy to be applied to a pathology of the subject.

30. The computer readable storage medium of claim 29 wherein the biological parameter value is indicative of radiosensitivity or a proliferation of the pathology.

31. (canceled)

32. An apparatus comprising:
a therapy planning system which establishes a characteristic of successive therapies applied to a subject as a function of a desired biological parameter value of the subject, a subject specific biological parameter history indicative of a pathology of the subject, and a pathology model which models response of the pathology to a therapy;
a therapy device operatively electrically connected to the therapy planning system so as to receive the established characteristic, and wherein the therapy device applies a therapy according to the established characteristic.

33. (canceled)

34. The apparatus of claim 32 wherein the characteristic includes a dose.

35. The apparatus of claim 34 wherein the characteristic includes a type of therapy.

36. The apparatus of claim 35 wherein the type of therapy includes at least one of a radiation and a chemical therapy.

37. The apparatus of claim 32 wherein the subject specific biological parameter history includes information from a functional imaging examination of the subject.

38. (canceled)

39. A method comprising:
obtaining data representative of a measured response of a patient population to an applied therapy, the data including a first measured biological parameter value, the applied therapy, and a second measured biological parameter value representative of a response to the applied therapy, wherein the first and second measured biological parameter values are obtained from functional imaging examinations of members of the subject population;
storing the data in a computer readable storage medium;
making the data available to a therapy planning system over a computer network.

40. (canceled)

41. (canceled)

42. The method of claim 39 wherein the data includes the measured responses of each of a plurality of members of the patient population to an applied therapy.

43. (canceled)
